(19) United States
(12) Patent Application Publication
(10) Pub. No.: US 2009/0192309 A1
(43) Pub. Date: Jul. 30, 2009

(54) PROCESS FOR PREPARING TRIALLYL CYANURATE

(75) Inventors: Volker Häfner, Langenselbold (DE); Helmut Suchsland, Rodenbach (DE); Peter Werle, Gelnhausen (DE)

Correspondence Address: LAW OFFICE OF MICHAEL A. SANZO, LLC, 15400 CALHOUN DR., SUITE 125, ROCKVILLE, MD, 20855, US

(73) Assignee: Evonik Degussa GmbH, Essen (DE)

(21) Appl. No.: 12/309,399

(22) PCT Filed: Jun. 25, 2007
(86) PCT No.: PCT/EP2007/056316

§ 371 Date: Jan. 16, 2009

(30) Foreign Application Priority Data
Jul. 21, 2006 (DE) 10 2006 034 247.7

(51) Int. Cl. C07D 251/34

(52) U.S. Cl. 544/192

(57) ABSTRACT

The invention relates to an improved process for preparing triallyl cyanurate (TAC) by reacting cyanuric chloride with allyl alcohol in the presence of an alkali metal acid acceptor and in the absence of an organic solvent other than allyl alcohol. According to the invention, TAC is obtained in over 99% purity with an APHA colour number below 10 in a yield of over 90% when 3.9 to 6.0 mol of allyl alcohol and 3.0 to 3.2 equivalents of acid acceptor are used per mole of cyanuric chloride, cyanuric chloride and acid acceptor are added simultaneously or successively to anhydrous or at least 50% by weight aqueous allyl alcohol, and the reaction is performed in one or more stages at a temperature in the range of −5 to +50° C.

The present invention relates to a process for preparing triallyl cyanurate (2,4,6-tris(allyloxy)-s-triazine), referred to hereinafter as TAC for short, in high yield and high purity, including an APHA number of less than or equal to 10.

Triallyl cyanurate (TAC) is a trifunctional monomer which is especially versatile in polymer chemistry and has three reactive allyl double bonds—see Kirk-Othmer, Vol. 2, p. 123-127. Uses of TAC include as a crosslinking component in the preparation of alkyd resins, polyurethanes, polyesters, and as a comonomer in vulcanization processes, and also as an adhesion promoter in rubber-latex mixtures for tyre cord. Moreover, TAC serves as a curing medium for a wide variety of different polymers; for example, copolymers of TAC with methacrylates give rise to glass-like substances with excellent optical and mechanical properties, as required for the production of high-quality optical glasses.

For many of the possible uses mentioned, very pure TAC is required, which also features a very low degree of discolouration, expressed as the APHA colour number, which should as far as possible not exceed the value of 10.

It is known that TAC is obtained by reacting cyanuric chloride with allyl alcohol in the presence of an acid acceptor, preferably of an alkali metal hydroxide. The reaction can be performed in the presence or absence of an organic solvent. The processes known to date are afflicted by various deficiencies, for instance too low a yield, inadequate purity or excessively complicated process technology for the preparation and isolation of the TAC.

U.S. Pat. No. 2,510,564 describes a process for obtaining TAC by adding cyanuric chloride to a suspension of sodium carbonate in 90% allyl alcohol (molar ratio 1:3.0:13.51 at temperatures up to 40° C., with subsequent heating up to 80° C.). In one modification of this method, powdered sodium hydroxide is used as the acid acceptor and the reaction is performed at room temperature. In both cases, it is necessary to filter off sodium chloride formed. What are obtained are opalescent to cloudy reaction products whose purity, at below 90%, leaves a great deal to be desired. The yields reported in the examples (85% and 76%) are likewise unsatisfactory.

In the processes of U.S. Pat. Nos. 2,631,148 and 3,644,410, the reaction is performed in the presence of toluene at below 10° C. and from 50 to 80° C. respectively. Even though both processes provide acceptable results with regard to product yield and purity, the performance of the process is complicated. The use of an organic solvent is not unproblematic, and makes the process more expensive through the equipment required and the energy input for the complete removal of the solvent from the aqueous and TAC-containing phase.

U.S. Pat. No. 3,635,969 teaches a process by which the reaction of cyanuric chloride with allyl alcohol and sodium hydroxide solution is effected in the absence of another organic solvent apart from the allyl alcohol reactant. The molar use ratio of cyanuric chloride to allyl alcohol to sodium hydroxide is 1:4.5:3.3, the sodium hydroxide concentration 40±0.5% by weight. The reactants are added to 70% aqueous allyl alcohol while maintaining a reaction temperature of 15±3° C. and a weakly alkaline pH. Disadvantages of this process, which permits the preparation of TAC with an APHA number around 10 in about 90% yield, are:

long addition and post-reaction times—for example around/above 10 hours overall—lead to low space-time yields;
the phase separation is time-consuming and the formation of emulsions formed entails additional technical measures, for instance those of coalescers;
after the distillative dealcoholization of the TAC-containing phase, a purification/filtration step, if appropriate with use of activated carbon, is required to remove flocculations.

In the reworking of the process of U.S. Pat. No. 3,635,969, the applicant of the present application also found that, in the workup of the aqueous reaction phase and wash phases for the purpose of recovering the allyl alcohol excess, there is contamination of the allyl alcohol with ammonia. Reuse of such a contaminated allyl alcohol in the TAC preparation results in the formation of triazine compounds which cannot be washed out and hence to an increase in the APHA colour number and corresponding reduction in the TAC quality.

It is an object of the present invention to improve the process known from U.S. Pat. No. 3,635,969, in order to at least partly remedy the disadvantages indicated. The process should also permit TAC to be prepared on the industrial scale with a yield of over 90%, based on cyanuric chloride, and an APHA colour number of below 10 as far as possible, and allow recovered allyl alcohol to be reused without reducing the quality of the TAC.

A process has been found for preparing triallyl cyanurate (TAC) having an APHA number of less than or equal to 10 by reacting cyanuric chloride with allyl alcohol in the presence of an alkali metal acid acceptor and in the absence of an organic solvent other than allyl alcohol, removing the salt formed by adding water and subsequent phase separation, extractively washing the organic phase with water and distillatively removing water and allyl alcohol from the TAC-containing organic phase, which is characterized in that 3.9 to 6.0 mol of allyl alcohol and 3.0 to 3.2 equivalents of acid acceptor are used per mole of cyanuric chloride, cyanuric chloride and acid acceptor are added simultaneously or successively to anhydrous or at least 50% by weight aqueous allyl alcohol, and the reaction is performed in one or more stages at a temperature in the range of −5° C. to +50° C.

Surprisingly, it has been found that a reduction in the amount of acid acceptor in the molar use ratio of the reactants compared to the processes known to date allows the reaction to be performed in a shorter time and without the tightly restricted temperature control required to date, and additionally allows TAC to be obtained with a relatively low APHA colour number, i.e. below 10. This allowed the space-time yield to be increased and TAC to be obtained in over 99% purity in yields over 90%. Surprisingly, the TAC-containing organic phase and the aqueous phase can additionally be separated from one another rapidly and without any problem after the reaction and the scrubbing, and recovered allyl alcohol can be reused without reducing the quality of the TAC.

The process according to the invention is technically simple to perform and requires a lower level of technical complexity compared to the process known to date, since phase separation problems and a filtration step do not occur.

In the process according to the invention, alkali metal compounds which are suitable as acid acceptors can be used. Essentially, they are thus oxides, hydroxides of alkali metals. Sodium hydroxide is particularly preferred as an acid acceptor. In principle, the acid acceptor can be introduced into the reaction mixture in pulverulent form or in the form of an aqueous solution or suspension.

The acid acceptor is preferably used in the form of an aqueous solution, especially sodium hydroxide solution. While the concentration of the sodium hydroxide solution used in the process known to date had to be very tightly restricted, the concentration in the process according to the invention is less critical; typically, the concentration will be between 30 and 50% by weight of NaOH, preference being given to a maximum concentration, i.e. in particular one around 50% by weight, since the amount of aqueous phase in the reaction mixture can be kept at a low level in this way.

According to the invention, 3.9 to 6.0 mol of allyl alcohol and 3.0 to 3.2 equivalents of acid acceptor are used per mole of cyanuric chloride. However, preferably 4.9 to 5.2 and in particular 3.05 to 3.10 equivalents of acid acceptor are used per mole of cyanuric chloride. In the case of use of sodium hydroxide solution as the acid acceptor, the numerical values specified correspond directly to the molar use ratio of the reactants.

The temperature range selected for the reaction is between −5° C. and +50° C., preferably between 0 and 40° C.; in a one-stage version, particular preference is given to a temperature range of +5° C. to +30° C. The reaction can be performed either isothermically at low temperature or semi-adiabatically with single-stage or multistage raising of the temperature. Preference is given to a two-stage process: here, the reaction in the first stage is performed up to a conversion of 65 to 80% at low temperature, for example at 0 to +15° C. and preferably +5 to +10° C.; in the second stage, the reaction is continued up to a conversion of essentially 100% at elevated temperature, preferably at +30 to +40° C.

The reactants cyanuric chloride and acid acceptor, especially sodium hydroxide solution, are introduced in any way into the allyl alcohol initially charged in excess, which may contain up to 59% by weight of water. Preference is given to using aqueous allyl alcohol having an allyl alcohol content of 75 to 90% by weight. The acid acceptor, which is preferably used in the form of an aqueous solution, can be added to a mixture of allyl alcohol or aqueous allyl alcohol and the total amount of cyanuric chloride. Alternatively, it is also possible to introduce cyanuric chloride and the acid acceptor simultaneously or with a certain initial feed of the cyanuric chloride into the initially charged, anhydrous or aqueous allyl alcohol, or a mixture comprising allyl alcohol and at least some of the cyanuric chloride. In a further but less preferred alternative, cyanuric chloride is introduced into a mixture of allyl alcohol and aqueous acid acceptor solution. In a particularly preferred embodiment of the process, sodium hydroxide solution, with and without simultaneous addition of cyanuric chloride, is introduced into a mixture of cyanuric chloride and allyl alcohol and optionally a little water.

To suppress hydrolytic side reactions which therefore reduce the yield, the reactants are combined sufficiently rapidly that the addition time, which corresponds to the greatest part of the overall reaction time, is kept to a minimum. The entire reaction time, i.e. that for the combination of the reactants and the post-reaction, is preferably not more than 3 hours and preferably less than 2 hours, in particular 1 to 1.5 hours.

In view of the high reaction enthalpy, in order to achieve the short reaction times, intensive cooling, for instance cooling using cooling brine, is required. On the industrial scale, the heat is removed particularly efficiently through an external cooling circuit with a suitable heat exchanger as well as a circulation pump.

As already detailed, the excess of acid acceptor is restricted to minimal values in the process according to the invention. In principle, the excess can be reduced to zero, but a minimal excess is useful with regard to the minimization of the post-reaction time. Only a very small excess of acid acceptor is found to be advantageous in two ways in the process according to the invention: firstly, the decomposition of the hydrolysis-sensitive TAC is suppressed, so that only insignificant yield losses, if any, occur; secondly, owing to the low alkalinity of the aqueous reaction phase and of the washing solutions in the distillative recovery of the excess of allyl alcohol used, there is no contamination thereof with ammonia as a result of hydrolytic cleavage of triazine compounds present in the aqueous phases.

After the reaction has ended, just sufficient water is added to the reaction mixture that the precipitated chloride goes back into solution. After the stirrer has been switched off, two phases form within a very short time: an upper organic phase which comprises virtually all of the TAC and a lower phase which comprises the salt. Typically, the phases are separated virtually instantaneously or within a few minutes, and give rise to a sharp separation line without formation of a crud layer. After removal of the aqueous phase, the organic phase is washed at least once, preferably two to three times, with water, in order to deplete the content of allyl alcohol and to wash out salt residues. The washing-out is effected preferably at temperatures around 30° C., which can be established easily under the process conditions. If appropriate, preheated water can also find use for the maintenance of the washing temperature of about 30° C. The washing of the organic phase can be performed batchwise or else continuously in a customary extraction apparatus. After the wash, the organic phase generally still contains about 2 to 7% allyl alcohol and 1 to 3% water. The volatile constituents mentioned are, after addition of a suitable polymerization inhibitor, typically a hydroquinone derivative, distilled off gently at elevated temperature and under reduced pressure. The TAC obtained as the bottom product in yields of significantly above 90% is a water-clear liquid having a purity of at least 99.5%, an APHA number of 0 to 10, preferably 0 to 5, and a solidification point of equal to or greater than 27° C. Allyl alcohol present in the combined aqueous phases of the reaction and the wash is preferably recovered therefrom as a 60 to 73% azeotrope with water, supplemented with 100% allyl alcohol and fed to a subsequent batch.

The examples which follow illustrate the process of the invention without restricting it. In a series of comparative experiments, the influence of the size of the sodium hydroxide excess, the reaction temperature and the post-reaction time on the cleavage caused by hydrolysis and the associated decomposition of the TAC formed was examined: it follows from this that, in the case of the inventive, very small excess of acid acceptor, the degradation rate of TAC is significantly lower than when using the excess mentioned of acid acceptor (sodium hydroxide solution) in the process known to date. In view of this finding, it is surprising that the significance of a very low excess of acid acceptor has not already been recognised before. The APHA number was measured according to EN ISO 6271-1:2004 (D).

### EXAMPLE 1

A coolable reaction vessel was initially charged with 354 g (5.0 mol) of 82% by weight allyl alcohol and cooled to 10° C. Thereafter, 184.5 g (1 mol) of cyanuric chloride were added, and 3.09 mol of 50% by weight sodium hydroxide solution were added dropwise with intensive stirring and cooling within 60 minutes, in the course of which the temperature was kept at 9 to 10° C. until 75% of the sodium hydroxide solution had been consumed. Thereafter, the cooling medium was removed and the remaining alkali was added rapidly, so that the temperature rose to 40° C. The mixture was stirred at 40° C. for another 15 minutes, in the course of which complete conversion was achieved according to analytical monitoring. Subsequently, 335 ml of water were added and the precipitated sodium chloride was brought into solution by adding water. After the stirrer had been switched off, two phases with a sharp separation line formed immediately. The upper organic phase was washed twice with 200 ml each time of water, which reversed the phases, and the TAC-containing phase was removed as the lower phase. The TAC-containing phase was stabilized with 100 ppm of hydroquinone monomethyl ether and, after being transferred into a rotary evaporator, dealcoholized at 90° C. in a water-jet vacuum. This gave 231.7 g of triallyl cyanurate of melting point 27° C., corresponding to a yield of 93.0%. The APHA number was determined to be 5, the purity to be 99.9%.

### EXAMPLE 2

The procedure was as in Example 1, except that the proportion of the sodium hydroxide solution metered in at 10° C. was increased to 80 l and the metering time correspondingly to 65 minutes. The remaining alkali was added rapidly and without cooling, in the course of which the temperature rose to 30° C. After 15 minutes of post-reaction time and the workup specified in Example 1, 235.5 g of triallyl cyanurate (=94.5% yield) were obtained in a purity above 99.9% and with an APHA colour number of zero. 164.0 g of 70% allyl alcohol, which contained less than 50 ppm of ammonia, was distilled off from the combined aqueous phases under virtually azeotropic conditions and used again (see Example 3).

### EXAMPLES 3 TO 6

Example 2 was repeated, except that the 60 to 70% by weight aqueous allyl alcohol which had been recovered essentially as an azeotrope from the preceding example in each case was supplemented to 5.0 mol with 100% by weight allyl alcohol and initially charged. Yield, purity and APHA number were virtually identical in Examples 3 to 6 and corresponded essentially to the values of Example 2; the APHA number was always significantly below 10.

### EXAMPLE 7

A brine-cooled reaction vessel was initially charged with 290.4 g of pure allyl alcohol which were cooled to −5° C. Thereafter, 184.5 g of cyanuric chloride were stirred in and the addition of 50% sodium hydroxide solution was commenced. Within 60 minutes, a total of 248.1 g (3.10 mol) of sodium hydroxide solution were metered in such that the temperature did not rise above 0° C. Subsequently, the mixture was allowed to continue to react without cooling for another approx. 30 minutes until complete conversion had been achieved. Thereafter, the mixture was warmed to 30° C.; to dissolve the precipitated sodium chloride, 409 ml of water preheated to 30° C. were added and, after the stirrer had been switched off, the phases which form within a few minutes were separated. The organic phase was removed, washed twice with 200 ml each time of water and, after stabilization with 100 ppm of hydroquinone monomethyl ether, freed of the volatile constituents on a rotary evaporator under reduced pressure. This gave 240.5 g (corresponding to a yield of 96.5%) of pure triallyl cyanurate with an APHA number of 0.

### EXAMPLE 8

A jacketed stirred reactor with an external heat exchange circuit consisting of cooler and circulation pump was initially charged with 119.0 kg (2049 mol) of allyl alcohol and 26.5 kg of water; 75.0 kg of cyanuric chloride were then added with stirring. Thereafter, the cooling circuit charged with brine was put into operation and the circulating mixture was cooled to 8° C. The addition of a total of 101.0 kg=66.2 l (1262 mol) of 50% by weight NaOH solution was then commenced, in the course of which the reaction temperature was kept at 9 to 14° C. until 54 l had been consumed.

Subsequently, the cooling circuit was shut down and the remaining sodium hydroxide solution was allowed to flow in within the shortest possible time. In the course of this, the internal temperature of the reactor rose to 35° C. The total introduction time was about 70 minutes. To complete the conversion, stirring was continued for another 20 minutes; 140 l of water were then added to the solution of the precipitated sodium chloride. After the stirrer had been switched off, two clear phases formed within a few minutes, which were separated by means of a separating vessel. The organic phase was recycled into the reactor and washed twice with 80 l each time of water. After the second extraction, the washed TAC still contained 2.1% water and 6.5% allyl alcohol. To remove the volatile fractions, the product, after stabilization with 100 ppm of hydroquinone monomethyl ether, was fed through a falling-film evaporator, which distilled off the low boilers at 50 mbar and 100° C. This gave 95.1 kg of triallyl cyanurate, corresponding to a yield of 93.9%; purity of the TAC 99.9%, APHA number 0 to 5.

### EXAMPLE 9

The reaction apparatus described in the preceding example, which had additionally been equipped with a metering unit suitable for pulverulent bulk material, was initially charged with 145.5 kg of 81.8% by weight allyl alcohol (2049 mol) together with 15 kg (81.3 mol) of cyanuric chloride, and the mixture was cooled to 8° C. Cyanuric chloride and the sodium hydroxide solution present in a small excess (50% strength by weight) were then metered in simultaneously under quantitative control continuously via the external heat exchanger circuit with intensive stirring and cooling, in the course of which the reaction temperature was kept at 9 to 10° C. Once the further addition of 60.0 kg (325.5 mol) of cyanuric chloride and 56 l (85.4 kg; 1061.7 mol) of 50% by weight sodium hydroxide solution was complete, the cooling was shut down and the remaining alkali of 10.2 l (15.6 kg; 194.5 mol) was allowed to flow in as rapidly as possible. Thereafter, the temperature rose to 30° C. The mixture was stirred at this temperature for a further 15 minutes; subsequently, workup was effected in the manner described in Example 8. This gave 95.8 kg of pure triallyl cyanurate, corresponding to a yield of 94.6%. The product had an APHA number of below 10.

### EXAMPLE 10

The procedure was as in Example 9, except that only 10% of the 75.0 kg of cyanuric chloride used were initially charged together with the allyl alcohol. After cooling to 8° C., the simultaneous addition of cyanuric chloride and 50% by weight sodium hydroxide solution in a molar ratio of 1:3.10 was commenced, in the course of which the internal temperature of the tank was kept at 9 to 10° C. Just before the end of the parallel metered addition, the reaction temperature was allowed to rise to 15 to 17° C. by appropriate regulation of the cooling. Once all of the cyanuric chloride had been added, the cooling was removed and the remaining sodium hydroxide solution was allowed to flow in rapidly, while the temperature rose further to 30 to 32° C. The total reaction time until the end temperature was attained was approx. 80 minutes. After a further 15 minutes of post-reaction time, workup was effected as described above. Yield and product quality do not differ from the preceding example.

### EXAMPLE 11

The procedure was as in Example 9, except that 50% of the cyanuric chloride was initially charged with the 81.8% by weight allyl alcohol at 8° C. After the end of the parallel addition, the reaction temperature was initially maintained further at 10° C.; only after addition of approx. 80 l of the total amount of sodium hydroxide solution was the cooling shut down. The total reaction time until the end temperature of 30 to 35° C. had been attained was 75 minutes. The yield of triallyl cyanurate was 94.1%, the purity 99.9% and the APHA colour number 0 to 5.

### EXAMPLE 12

The procedure was according to Example 9, except that, for this purpose, the allyl alcohol recovered as a 60% solution from this example was reused and supplemented to the total amount of 5.0 mol/mol of cyanuric chloride with fresh allyl alcohol. This lowered the concentration of the allyl alcohol used to 78.9%. 95.0 kg were obtained, corresponding to a yield of 93.8% of triallyl cyanurate. The content was 99.7%; the APHA number was measured at 10.

### EXAMPLE 13

To illustrate the improvement of the process according to the invention over the prior art process, the influence of the sodium hydroxide excess, the post-reaction temperature and the post-reaction time on the cleavage, caused by hydrolysis, of the TAC formed and its degradation rate as a function of the parameters mentioned were determined in a series of comparative experiments.

In each case, a model mixture of TAC, allyl alcohol, water and NaCl prepared according to Example 2 was used, with the proviso that the reaction was performed without sodium hydroxide excess (3.0 mol of 50% by weight NaOH per mole of cyanuric chloride), and that, after the reaction had ended, no dilution water was added. The TAC content in the mixture was in each case 64.0 to 64.2%. After the desired post-reaction temperature had been set, these model mixtures were admixed with a certain amount (corresponding to the desired excess) of 50% by weight sodium hydroxide solution, and stirred at constant temperature for several hours. Samples were taken at certain time intervals, and their TAC content was compared with the TAC content of the starting sample (zero sample) of the model mixture. The post-reaction temperature, the added NaOH excess (mol per mole of cyanuric chloride) and the TAC degradation rate (%) after 30, 60, 120 and 180 minutes of the experiments follow from the table. The results demonstrate the harmful influence of relatively high NaOH excesses on the decomposition of the TAC formed.

TABLE

| | NaOH excess | Temper- | TAC degradation (%) | | | |
|---|---|---|---|---|---|---|
| Exam- | (mole of NaOH per mole | ature | after minutes | | | |
| ple No. | of cyanuric chloride) | (° C.) | 30 | 60 | 120 | 180 |
| 12.1 | 0.09 | 30 | 0.1 | 0.2 | 0.4 | 0.5 |
| 12.2 | 0.09 | 50 | 0.6 | 1.6 | 2.5 | 3.1 |
| 12.3 | 0.15 | 40 | 0.2 | 0.5 | 1.5 | 2.8 |
| 12.4 | 0.30 | 50 | 4.2 | 10.0 | 22.7 | 32.4 |
| 125 | 0.75 | 30 | 2.5 | 4.7 | 11.8 | 17.0 |

N.B.: Examples 12.1 to 12.3 have an inventive NaOH excess; the NaOH excess of Example 12.4 corresponds to that of U.S. Pat. No. 3,635,969.

1-10. (canceled)

11. A process for preparing triallyl cyanurate (TAC) having an APHA number less than or equal to 10 comprising:
a) reacting cyanuric chloride with allyl alcohol in the presence of an alkali metal acid acceptor and in the absence of an organic solvent other than allyl alcohol;
b) removing the salt formed by adding water and subsequent separation of aqueous and organic phases;
c) extractively washing the organic phase with water;
d) removing water and allyl alcohol from the TAC-containing organic phase by distillation;
wherein:
i) 3.9 to 5.0 mol of allyl alcohol and 3.0 to 3.2 equivalents of acid acceptor are used per mole of cyanuric chloride;
ii) cyanuric chloride and acid acceptor are added simultaneously or successively to anhydrous or at least 50% by weight aqueous allyl alcohol; and
iii) the reaction is performed in one or more stages at a temperature in the range of −5° C. to +50° C.

12. The process of claim 11, wherein said acid acceptor is an alkali metal hydroxide.

13. The process of claim 12, wherein said acid acceptor is sodium hydroxide.

14. The process of claim 13, wherein said sodium hydroxide is in the form of a concentrated aqueous solution.

15. The process of claim 11, wherein 4.9 to 5.2 mol of allyl alcohol and 3.0 to 3.15 equivalents of acid acceptor are used per mole of cyanuric chloride.

16. The process of claim 15, wherein 3.05 to 3.10 equivalents of acid acceptor are used per mole of cyanuric chloride.

17. The process of claim 11, wherein said reaction is performed at 0 to 40° C.

18. The process of claim 11, wherein said reaction is performed in two stages:
a) a first stage which is carried out at a temperature of 0 to +15° C. and in which 65%-90% of said triallyl cyanurate is produced; and
b) a second stage which is carried out at a temperature of 30° C. to 40° C. and in which up to 100% of said triallyl cyanurate is produced.

19. The process of claim 18, wherein said first stage is carried out at a temperature of +5° C. to +10° C.

20. The process of claim 11, wherein aqueous allyl alcohol with an allyl alcohol content of 75 to 90% by weight is used.

21. The process of claim 11, wherein the entire reaction time is limited to not more than 3 hours.

22. The process of claim 11, wherein the organic phase is washed at least once with water at a temperature of about 30° C.

23. The process of claim 11, wherein the allyl alcohol present in the aqueous phase is distilled off therefrom and recycled to a subsequent batch.

24. The process of claim 12, wherein 4.9 to 5.2 mol of allyl alcohol and 3.0 to 3.15 equivalents of acid acceptor are used per mole of cyanuric chloride.

25. The process of claim 24, wherein said reaction is performed at 0 to 40° C.

26. The process of claim 24, wherein the reaction is performed in two stages:
a) a first stage which is carried out at a temperature of 0 to +15° C. and in which 65%-90% of said triallyl cyanurate is produced; and
b) a second stage which is carried out at a temperature of 30° C. to 40° C. and in which up to 100% of said triallyl cyanurate is produced.

27. The process of claim 26, wherein said first stage is carried out at a temperature of +5° C. to +10° C.

28. The process of claim 27, wherein aqueous allyl alcohol with an allyl alcohol content of 75 to 90% by weight is used.

29. The process of claim 28, wherein the entire reaction time is limited to not more than 3 hours.

30. The process of claim 29, wherein the allyl alcohol present in the aqueous phase is distilled off therefrom and recycled to a subsequent batch.
